(19) United States
(12) Patent Application Publication
(10) Pub. No.: US 2003/0109867 A1
(43) Pub. Date: Jun. 12, 2003

(54) Ablation instrument and method for cutting, fragmenting and/or removing material

(75) Inventors: Peter Gluche, Ulm (DE); Stephan Ertl, Ulm (DE); Christian Lingenfelder, Ulm-Mahringen (DE); Erhard Kohn, Ulm/Lehr (DE)

Correspondence Address: Andrew R. Basile, YOUNG & BASILE, P.C., 3001 West Big Beaver Road, Suite 624, Troy, MI, 48084, US

(73) Assignee: GFD-Gesellschaft fur Diamantprodukte mbH, Ulm (DE)

(21) Appl. No.: 10/077,033

(22) Filed: Feb. 15, 2002

(30) Foreign Application Priority Data
Dec. 6, 2001 (EP) 01 12 9004.6

(51) Int. Cl. A61B 18/18

(52) U.S. Cl. 606/41; 606/28

(57) ABSTRACT

Ablation instrument is provided for cutting, fragmenting and/or removing material of an object, containing a carrier substrate, having at least one resistance-heated layer which is disposed on the substrate and made of doped diamond or diamond-like carbon (DLC) and also at least one electrical lead and at least one electrical discharge which are both electrically connected at different places by the resistance-heated layer.

The present invention relates to an ablation instrument and also a method for cutting, fragmenting and/or removing material. Methods of this type are required in order to remove material from an object or to cut into said object or to destroy it. In particular, but not exclusively, methods and ablation instruments of this type are required in the field of medicine, in particular in the field of cataract extraction, minimally invasive surgery or vitrectomy.

According to the state of the art, ultrasonic cutting elements, so called phacoemulsifiers, are used in order to destroy the optic lens. A sharp cutting wedge is hereby moved back and forth very rapidly by means of piezocrystal and consequently the material of the lens is cut into pieces. Alternatively a target is introduced into the lens via an applicator stick onto which a laser beam is directed. The laser beam now heats the target and this produces a vapour bubble in the material which destroys the material because of its explosive-like expansion in the vicinity of the bubble. This destroyed fragmented material can then be removed by suction for example through the applicator tube. Titanium or tantalum are thereby used as the target, short-term hotspots being achieved with a temperature of above 320° C. by irradiation of a suitable laser pulse.

These conventional systems are complex and very expensive not least because of the required laser system. In addition, the applicator sticks are constructed in a complex fashion and are complicated to handle. Their target is in addition very rapidly worn out.

It is therefore the object of the present invention to make available an ablation instrument and a method for cutting, fragmenting and/or removing material of an object, which enables a material treatment of this type in a simple, precise and economical manner.

This object is achieved by the ablation instrument according to claim 1 and the method according to claim 38. Advantageous developments of the instrument according to the invention and of the method according to the invention are given in the respective dependent claims.

According to the invention, the ablation instrument has at least one resistance-heated layer made of doped diamond or diamond like carbon. This heating layer is now supplied with a current via electrical leads and discharges, for example made of doped diamond, such as p⁺-diamond, amorphous silicon, Ti, W, Pt, Au, TiW, WC, TiC, TiN, Si, Cu, Be, Fe, Al, Ni, C, Sn, Ba, Cr or refractory metals and it serves for producing very high temperatures. The heating layer is thereby disposed on a substrate so that, for example via a mounting, said layer can be brought into direct contact with the object to be treated or into its vicinity. There is thereby understood by object any processable material, including human, animal or plant tissues. As long as the material is not directly contacted, a liquid can be introduced in addition between the heating layer and the material. The heating layer and hence the liquid contacted by the latter or the material contacted with the heating layer are now heated temporarily to very high temperatures by means of short current pulses so that vapour bubbles are formed which, as also in the state of the art, lead to an explosive-like bursting or fragmentation of the material.

A resistance-heated layer made of doped diamond or DLC is suitable for the ablation instrument according to the invention, particularly because of its outstanding chemical and physical properties. These are in particular its extremely high thermal conductivity in combination with an extremely low heat capacity, high mechanical stability and also low thermal expansion. These properties are present in the case of diamond extensively independently of the electrical conductivity which can be adjusted via the dopant concentration.

Diamond therefore shows a high thermal loadability which also permits homogenous heating even of large heating elements also in the case of extremely high temperatures and short heating times and at the same time prevents their thermal destruction. The energy introduced into the heating element is transferred to surrounding media in addition extremely rapidly because of the low heat capacity. Consequently, an effective, temporally extremely rapid heating of these media is possible. In addition, this enables direct conversion of electrical energy into heat energy, a very homogenous heat distribution on the heating element, no cavitation damage on the heating element caused by the bubble which is produced and also no thermal stress for the heating element despite the extremely rapid heating and cooling.

The use of a diamond heating element, which can be in addition insulated electrically by applying an insulating diamond layer on its surface and if necessary on the adjacent electrical leads, permits direct conversion of electrical energy into heat. Consequently the system-conditioned, loss-associated intermediate steps, such as for example with a laser (electrical energy—optical energy—heat) are avoided.

The mechanical hardness of the diamond surface in addition prevents the damage arising which occurs constantly in the case of the methods known from prior art by cavitation.

Advantageously, a thermally insulating flyer is disposed between the substrate and the resistance-heated layer in order to impede dissipation of the heat into the substrate and thus to improve the heatability of the resistance-heated layer and to increase the possible frequency of the individual heat pulses. In addition, one or more intermediate layers, especially made of nominally undoped diamond with a energy gap of approximately 5.45 eV, can be disposed between the thermally insulating layer and the resistance-heated layer in order for example to stop electrical leakage currents which flow at high temperatures through a thermally insulating substrate, for example made of SiO₂. In this case, diamond is advantageous because of its low thermal expansion since consequently thermally-conditioned mechanical tensions between the layers are minimised and detachment of the layers can be avoided. The combination of conductive and insulating diamond layers is particularly advantageous since then absolutely no thermally-conditioned mechanical tensions can occur. The intermediate layer can in addition serve for the purpose of improving the nucleation capacity of the thermally insulating layer for doped diamond or DLC. Furthermore, the resistance-heated layer and possibly also the adjacent electrical leads can have at least partially on their other side, which is orientated towards the material to be treated, an electrically insulating layer, preferably made of nominally undoped and hence insulating diamond which leads to the fact that the current is in fact restricted exclusively to the resistance-heated layer.

It is furthermore absolutely essential in the present invention that diamond surfaces are chemically and physically inert and hence bio-compatible and therefore are especially suitable for medical, for example intracorporal applications.

According to the invention, the resistance-heated layer can be supplied with individual cu rent pulses with frequencies up to approximately 50 kHz in contact with water. These current pulses can be repeated periodically or aperiodically. The limiting frequency for the current pulses is thereby of course dependent upon the surrounding medium, especially its viscosity and thermal properties.

It is advantageous in the present invent on that no complicated optical structural elements (lasers) or mechanically moving parts (phaco tip with a piezo drive) are used. Consequently, the probability of failure of the ablation instrument is also extensively ruled out. In the case of the ablation instrument according to the invention, power densities of above 700 kW per cm² can be converted since diamond has an extremely high mechanical stability, electrical conductivity, thermal conductivity and a low thermal coefficient of expansion. Because of the good thermal conductivity and the homogenous temperature distribution resulting therefrom, heating elements >0.1 mm², preferably 0.1 to 10 mm² very particularly preferred 0.1 to 5 mm² can be produced with which large bubbles can be produced. In total, a very reliable ablation instrument is thus offered which can be used even as a disposable instrument because of its dimensioning and cost.

If a thermal element which can also be made of diamond is integrated in the ablation instrument, for example directly into the heating layer, then control of the produced temperature, for example dependent upon the surrounding medium, is also possible. The heating element itself can thereby represent the thermal element because of the dopant concentration dependent activation energy and be monolithically integrated.

According to the invention, the configuration of the bubble produced can be determined by a pre-selected configuration of the resistance-heated layer. Also the temperature at which the bubble is produced can be specifically achieved advantageously close to or above the spinodalian temperature of the liquid used.

In the case of an ablation instrument which contains at least one resistance layer made of doped diamond or DLC (diamond-like carbon) which is disposed on a thermally insulating layer, the diffusion of the heat generated in the resistance-heated layer is significantly reduced and hence the efficiency of the heating layer is significantly improved. In contrast to the heating layers of the state of the art, the heating layer according to the invention is located neither in thermal contact with a large area diamond film nor with other heat sinks because of appropriate structuring. Lateral diffusion of the heat is prevented by measures based on techniques such as mesa etching or selective growth. Because of the vertical layer structure which has a thermal insulating layer, heat diffusion into the substrate is also, ruled out. The use of the insulating layer makes the thinning of the substrate, which is known from the state of the art, superfluous so that the heating layer according to the invention can be disposed on mechanically stable commercial substrates. The insulating layer has preferably a heat conductivity of below 1 W cm⁻¹ K⁻¹ at 300 K and can comprise materials such as for example silicon oxides, silicon nitrides, silicon oxynitrides and aluminium oxides. In order to ensure an adequate thermal insulation of the heating layer, the thickness of the insulating layer should be at least 0.25 μm and preferably in a range between 1 μm and 10 μm. The insulation layer is made particularly preferably of amorphous materials which have low thermal conductivities. The insulation layer has preferably no electrical conductivity or only low electrical conductivity.

The doped resistance-heated layer can be produced directly on the insulating layer, for example by means of MWPECVD or hot filament techniques. Alternatively, it is also conceivable to provide one or more intermediate layers between the resistance-heated layer and the insulating layer. In order to avoid lateral heat diffusion, the at least one intermediate layer should either be itself a thermal insulator or else be structured laterally.

As already explained, surfaces areas of the resistive heating element of 60×60 μm² to 10 mm², preferably 1 to 5 mm² can be produced with which a bubble can be produced. This is only possible with a resistance-heated layer made of diamond because of its above-described special electrical and thermal properties.

A preferred embodiment of the invention provides that the heat loss of the ablation instrument according to the invention is detected by temperature sensors which are integrated monolithically in the heating layer in order to ensure exact control of the ablation instrument. For this purpose a diamond region with a dopant concentration of >10⁷ cm⁻³ is used for the temperature sensor. The dopant concentration in the resistance layer of the heating element should on the other hand be at least approximately 10¹⁹ cm⁻³ in order to produce a temperature-independent electrical resistance. In the case of a lower dopant concentration and if a certain temperature dependence of the heating behaviour is taken into account, the ablation instrument can also function itself as a temperature sensor by means of choosing a suitable dopant concentration.

In the following, a few examples of the ablation instruments according to the invention and the methods according to the invention are given.

There are shown:

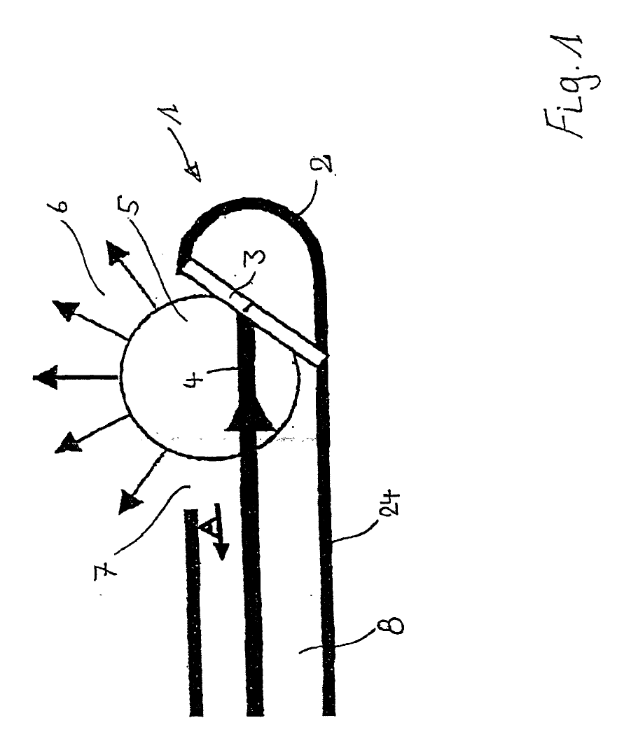

FIG. 1 a conventional ablation instrument;

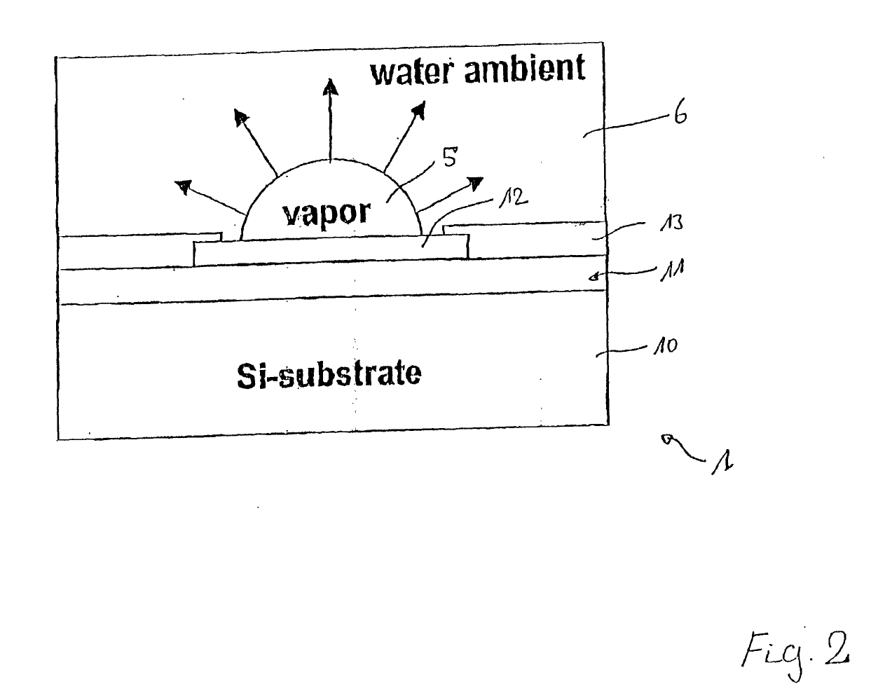

FIG. 2 the construction of an ablation instrument according to the invention;,

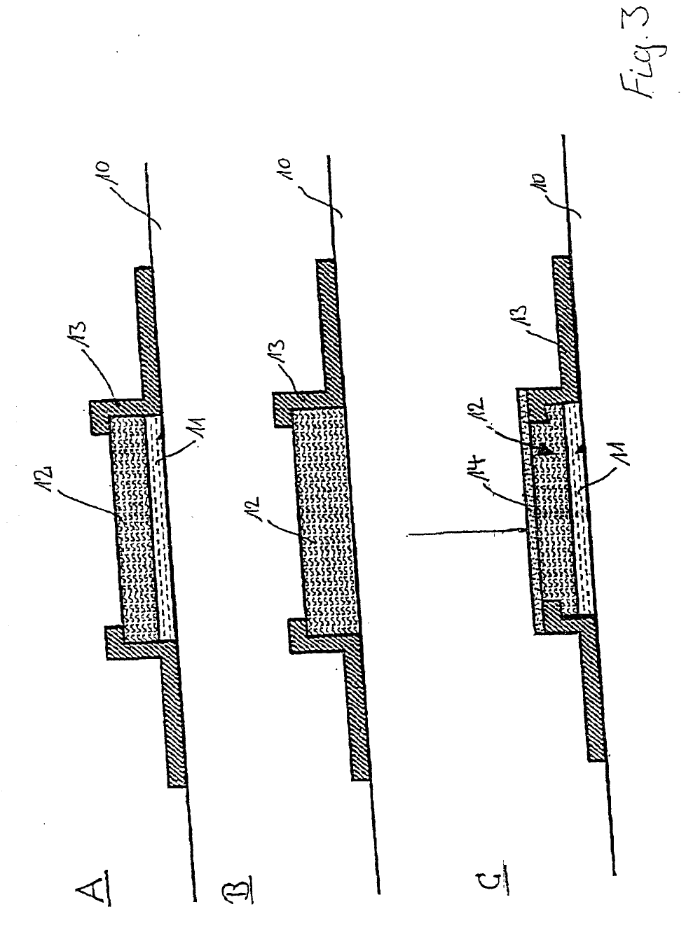

FIG. 3 various further ablation instruments;

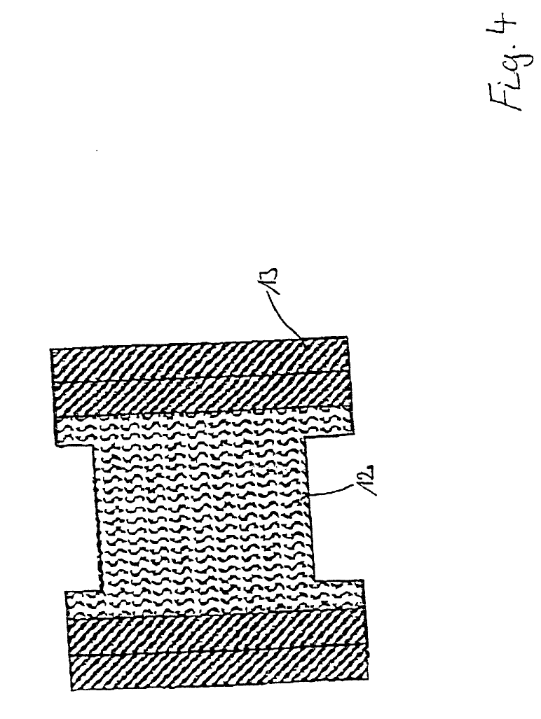

FIG. 4 a plan view of an ablation instrument;

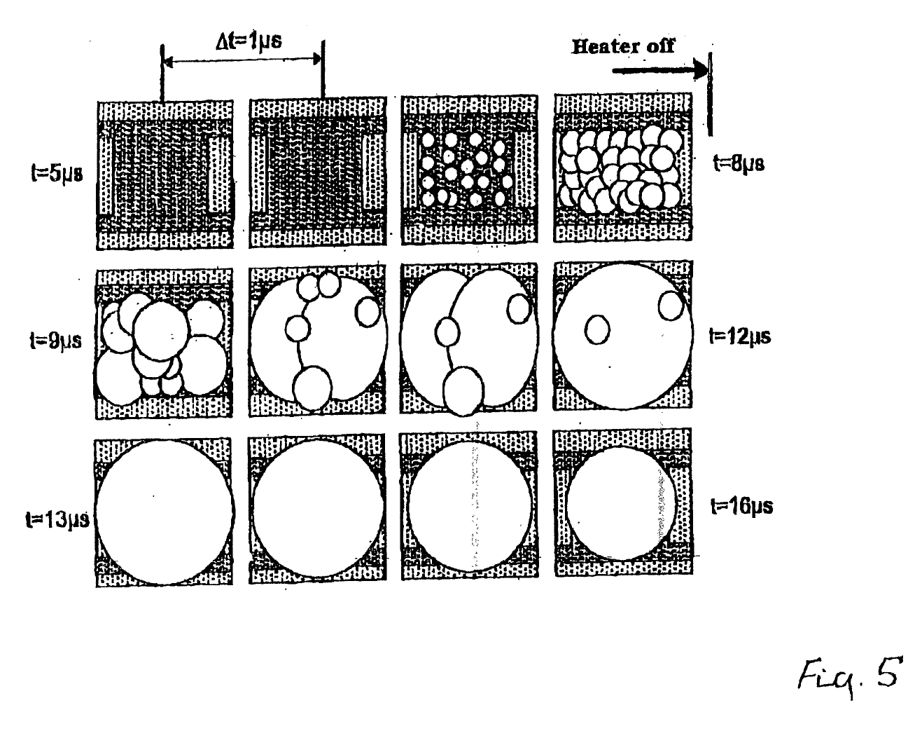

FIG. 5 the course of the bubble production with an ablation instrument according to the invention;

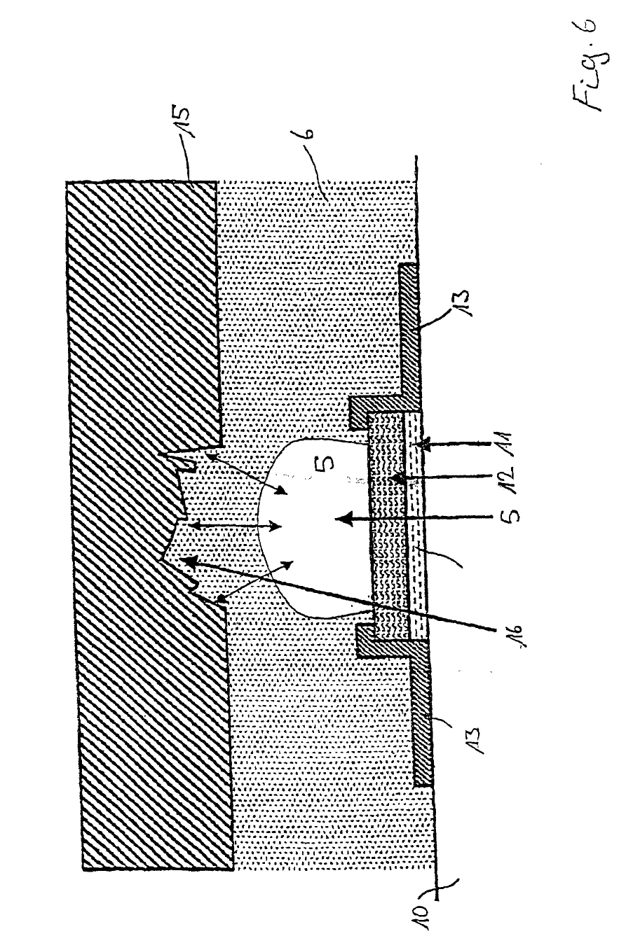

FIG. 6 to FIG. 13 various forms of ablation instruments according to the invention; and

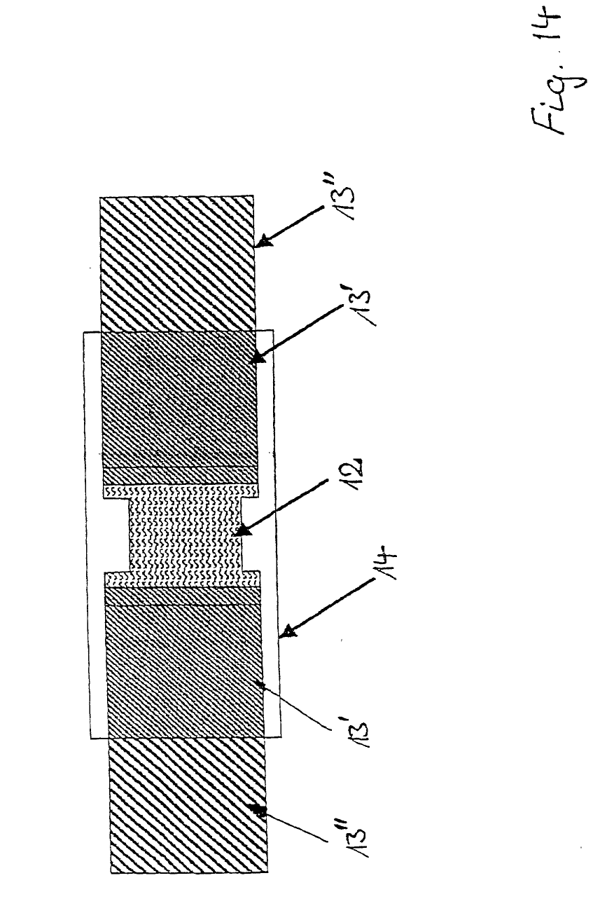

FIG. 14 a plan view of a further ablation instrument.

FIG. 1 shows an ablation instrument 1 according to the state of the art. This ablation instrument comprises an applicator tube in the form of a tube 2, which has a lateral opening 7 on the end illustrated in FIG. 1. Furthermore, a target 3 is disposed at an angle relative to the axial direction of the tube 2 and is made for example of titanium or tantalum. This tube 2 has a channel-like through-opening 8 which is formed by the external walls 24 of the tube 2 and communicates with the opening 7.

As is illustrated in FIG. 1, a laser bear 4 is aimed at the target 3 now in order to induce material destruction. The target 3 is in contact with a liquid or the surrounding tissue, for example a lens. By means of the laser beam 4, the target 3 is heated to a very high temperature, usually up to the spinodalian temperature of the surrounding liquid.

As a result of this, the medium 6 which is in contact with the target 3 is overheated and forms in an explosive manner a gas bubble 5 which expands in an explosive manner in the direction of the five arrows which are illustrated. This gas bubble transfers a shock wave to the medium 6 and as a result destroys the latter. Next, the ablated material is removed by suction in the direction of the arrow A through the opening 8.

FIG. 2 shows the structure of an ablation tool 1 according to the invention. This has a silicon substrate 10 on which a thermally insulating layer 11 and also a resistance-heated layer 12 is applied. The resistance-heated layer 12 is contacted via electrical contacts 13 (metallisations). Once again, the upper surface of the resistance-heated layer 12 made of diamond is in contact with the medium 6. If a current is now passed through the diamond layer 12 via the metallisation 13, then the diamond layer 12 heats up and in turn forms a gas bubble in the medium 6.

An advantage with the ablation instrument according to the invention is furthermore the good cleaning possibility because of the inertness of the active element since absolutely no tissue or hardly any tissue adheres.

In order to produce a layer structure illustrated in FIG. 2, a 3 μm thick SiO₂ layer 11 is deposited firstly on a silicon carbide or silicon substrate 10 by means of CVD technology. Also by means of a CVD method, a p⁺-doped diamond layer with a dopant concentration of 10²⁰ to 10²¹ cm⁻³ and a thick ness of 400 nm to 10 μm, preferably 1 to 3 μm is grown on the SiO₂ layer 11. The p⁺-doped diamond film functions as a resistance-heated layer 12.

In the partial figures A-C, FIG. 3 shows the construction of three different ablation tools according to the invention.

In FIG. 3A, a thermal insulation layer 11 made of SiO₂ is applied to a substrate 10 and on said layer a resistance-heated layer 12 made of doped diamond. The metallic contacts 13 extend from the resistance-heated layer 12 over the substrate 10.

In FIG. 3B the resistance-heated layer 12 is situated on the substrate and in FIG. 3C, on the resistance-heated layer 12 and on the parts of the metallic contacts 13 at the same level, there is applied an electrically insulating layer 14 made of intrinsic diamond or SiO₂. This electrical insulation layer 14 has only a low layer thickness in order that heating of the medium is nevertheless ensured reliably. Electrical insulators with good heat conductivity (for example diamond) are possible hereby as materials.

FIG. 4 shows a plan view of a resistance-heated layer 12 and of the electrical contact 13 for ablation tools as are shown in FIGS. 3A and 3B. It can be detected here that the resistance-heated layer 12 is constricted between the two contact layers and, compared to the smallest width of the resistance-heated layer 12, the contact faces between the metallisation 13 and the resistance-heated layer 12 are very wide and large-surface. Consequently the transition resistances between the resistance layer 12 and the contacts 13 are reduced so that heat production is effected essentially in the resistance-heated layer 12.

FIG. 5 shows the production of a gas or vapour bubble in water with an electrical power of 10 W in temporal sequence, the individual drawings following each other from left to right and from above to below. The temporal spacings between the individual drawings are 1 μsec, the first drawing beginning top left at a time t=5 μsec after the beginning of the current flow through the heater and the last drawing indicating bottom right the time at t=16 μsec. In this example, an electrical current is supplied in total for 8 μsec to the resistance-heated element 12, wherein the spinodalian temperature of tie surrounding liquid having been exceeded.

As can be detected in FIG. 5, small local gas bubbles are formed after 7 μsec after exceeding the spinodalian temperature, said gas bubbles combining into a large gas bubble up to after 12 μsec which forms its largest expansion state after 13 μsec. After that, the gas bubble 5 degenerates again.

This rapid formation and degeneration of the gas bubbles which is effected within μ-seconds enables a frequency of the gas bubble formation between 0 and 30,000 Hertz at an energy per pulse of 0-7 mJ. This corresponds to powers of 0-145 W. Therefore power densities of 0-210 kW/cm² were already measured with a surface of the heating element of 60×60 μm², a current pulse duration of 7 μsec add a frequency of 20 kHz.

Hence with respect to frequency, energy per pulse and power introduced into the surrounding medium and also power density, the ablation instrument according to the invention lies far above the conventional ultrasonic or laser methods.

FIG. 6 shows the use of an ablation tool according to FIG. 3A. When used for material removal from an object 15. In this case a medium 6, for example water, is introduced between the object 15 and the resistance-heated layer 12. This medium now forms the bubbles according to the invention and destroys, by means of the cavitative effect of this bubble, the surface of the object 15. Consequently, the surface of the object 15 is fragmented and removed. The fragments produced can then be removed by suction or be removed in another manner.

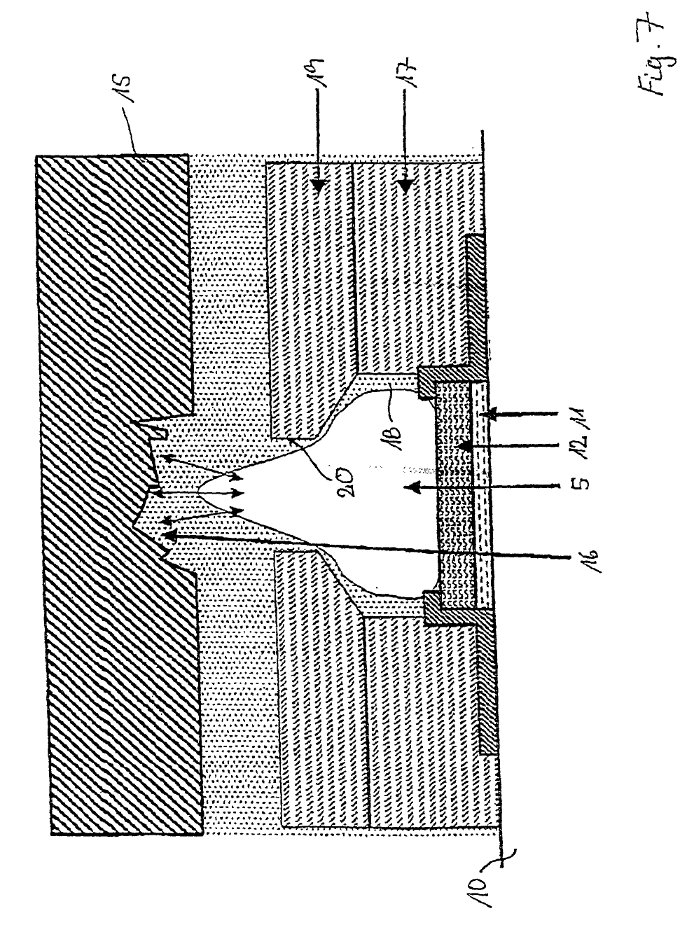

FIG. 7 shows an ablation tool which has a similar construction to that in FIG. 3A. In addition, a capillary system 17 is disposed above the resistance-heated layer 12 and transports here, in a vertical direction relative to the plane of the drawing, a medium onto the surface of the heating element 12 via a capillary 18.

Furthermore, a nozzle plate is disposed above the capillary system 17 through which nozzle plate the cross-section of the capillary is further narrowed and hence a narrow nozzle opening is formed which joins the capillary 18 and the surface of the resistance-heated layer 12 to the external side of the ablation tool. In this arrangement, the gas bubble 5 which is produced is centrifuged through the nozzle opening 20 formed in the nozzle plate 19, as a result of which the cavitative effect can be increased further on the object 15. In addition, it is possible by means of the capillary system always to supply adequate medium to be evaporated to the resistance-heated layer 12.

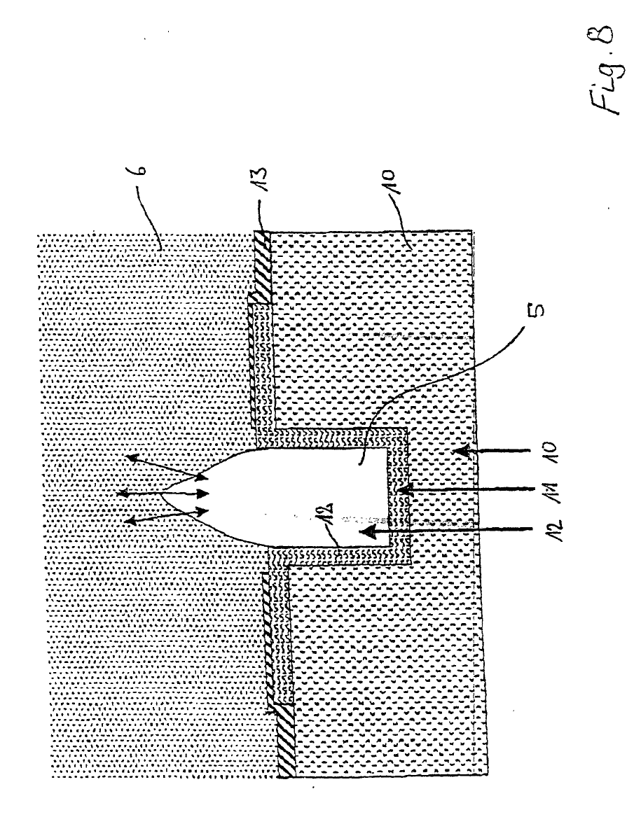

FIG. 8 shows a further ablation tool according to the invention in which the resistance-heated layer 12 has a three-dimensional configuration and forms a pot in the substrate 10. Consequently, the contact surface of the resistance-heated layer 12 with the medium 6 is enlarged and the resistance-heated laser 12 forms itself a nozzle-like opening on its upper side.

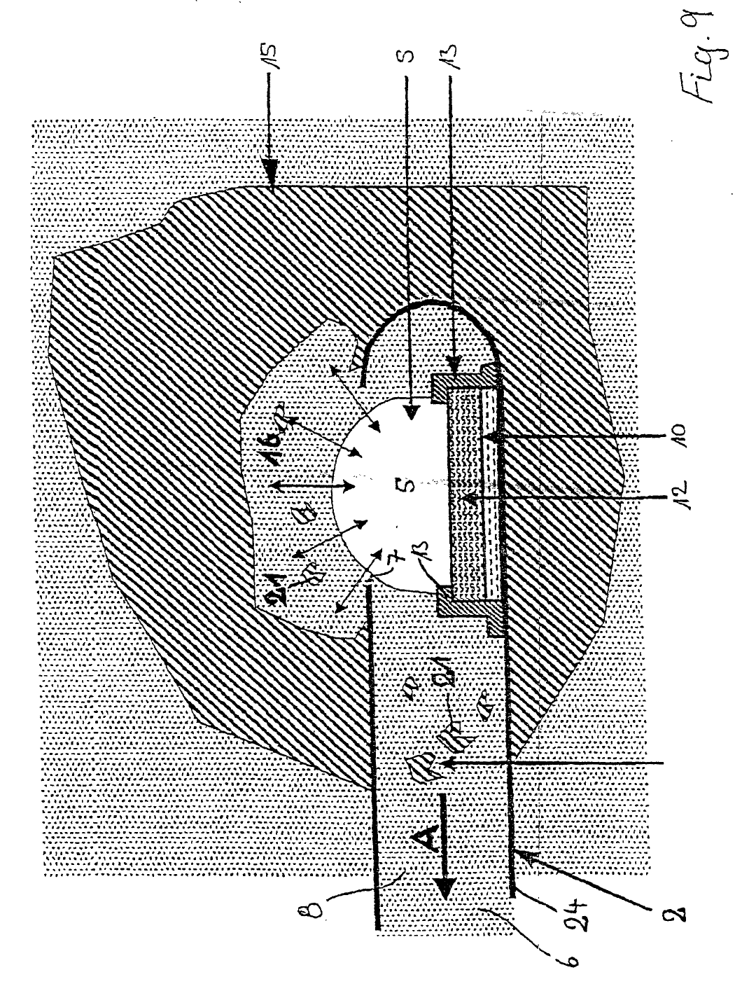

FIG. 9 shows the arrangement of an ablation tool according to FIG. 3A within an applicator stick 2. The resistance-heated layer is thereby disposed with its surface on one of the lateral walls 4 of the applicator stick 2 which is configured as a tube. Opposite this on the circumference of the applicator tube 2 there is an opening 7 in the wall 24. The vapour bubble 5 produced on the surface of the resistance-heated element 12 can now emerge through this opening and destroy the object 15. Material fragments 21 are removed in this region and a cavity 16 it formed in the object 15. The fragments 21 can thee be removed by suction through the interior space of the tube 2, which is configured as a suction channel 8, in the direction of the arrow A.

It should be mentioned here, that a cut can be introduced in the material 15 by the linear movement of the applicator tube 2 so that the device according to the invention is also suitable for cutting objects. This device can also be used in endoscopy.

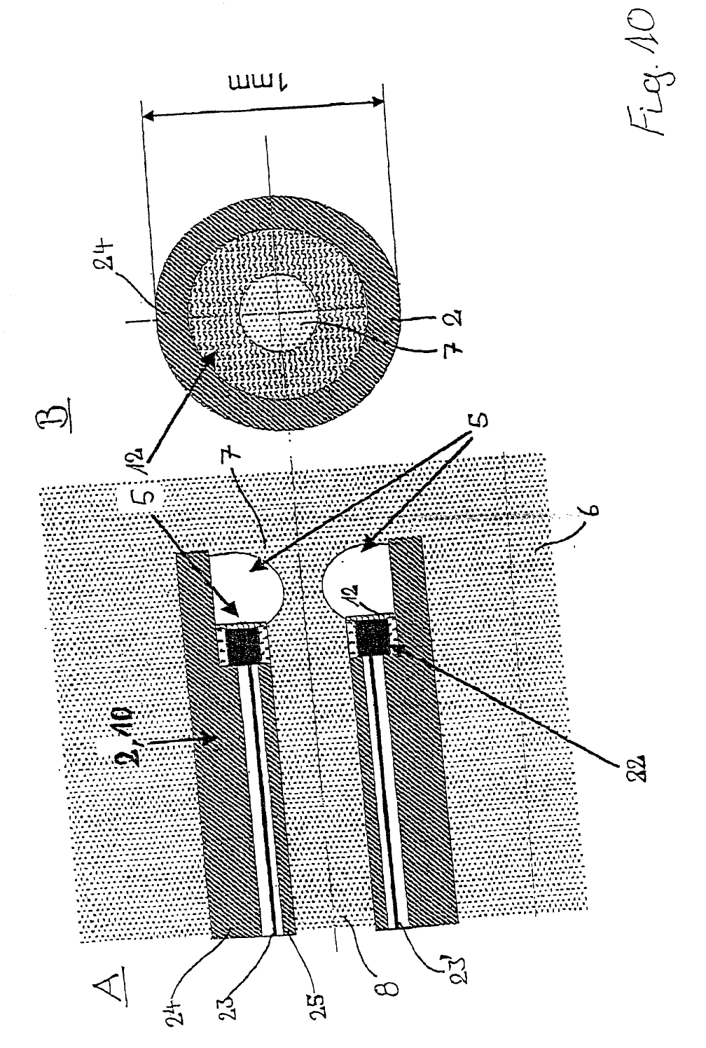

FIG. 10 shows a further example of an ablation tool in an applicator tube 2. The external well 24 of the applicator tube 2 forms the substrate at the same time. The applicator tube has, as can be seen in FIG. 10A, an opening 7 in the axial direction. FIG. 10B shows a cross-sectional view of this opening 7. As can be detected, surrounding the opening annularly there is disposed a resistance-heated layer 12 which can be contacted via a rear side contact 22 and an electrical lead 23 and an electrical discharge 23′. These wires 23, 23′ extend respectively within openings between the internal wall 25 and the external wall 24 of the applicator tube. The internal wall 25 is concentric and includes a cavity 8 as suction channel.

If the resistance-heated layer 12 is now supplied with an electrical current then a radially symmetrical gas bubble is formed, which in the course of further heating grows together and is expelled from the opening 7.

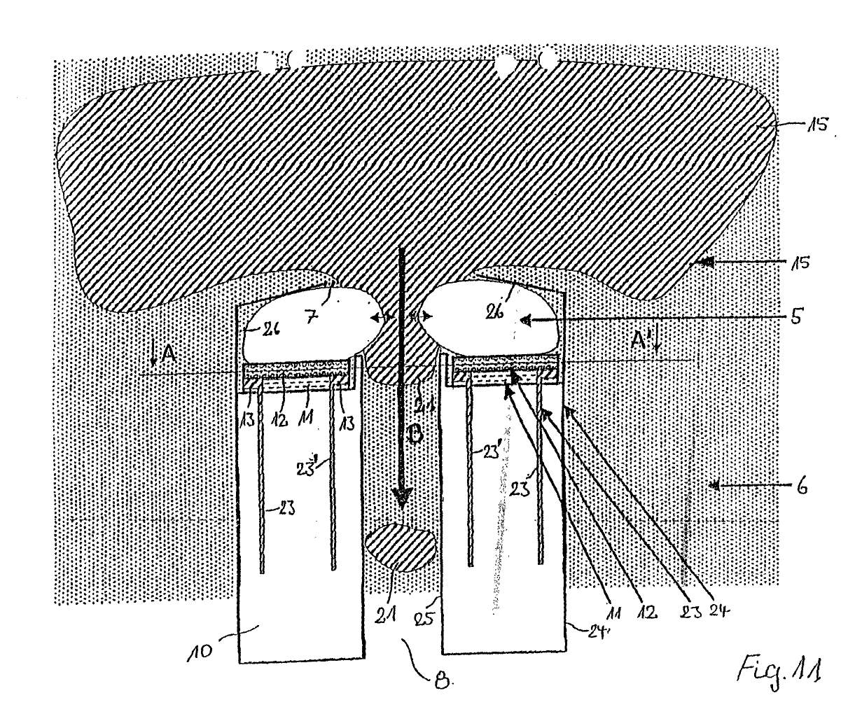

FIG. 11 shows a modification of an ablation tool as it is illustrated in FIG. 10. In contrast to FIG. 10, the external wall 24 now extends over the resistance-heated layer 12 and is angled over then inwardly in the direction of the axis of the tube 2. In this manner, the resistance-heated layer 12 is covered at a spacing, the external wall 24 leaving free a concentric opening 7 for discharge of the gas bubble.

It is illustrated in FIG. 11 how the application tool according to the invention can also be used for removing a flexible material 15. For this purpose, this material 15 is sucked into the opening 7 where the produced gas bubbles 5 respectively cut off small fragments 21. These are then sucked in the direction of the arrow B through the suction channel 8 which extends axially centrally.

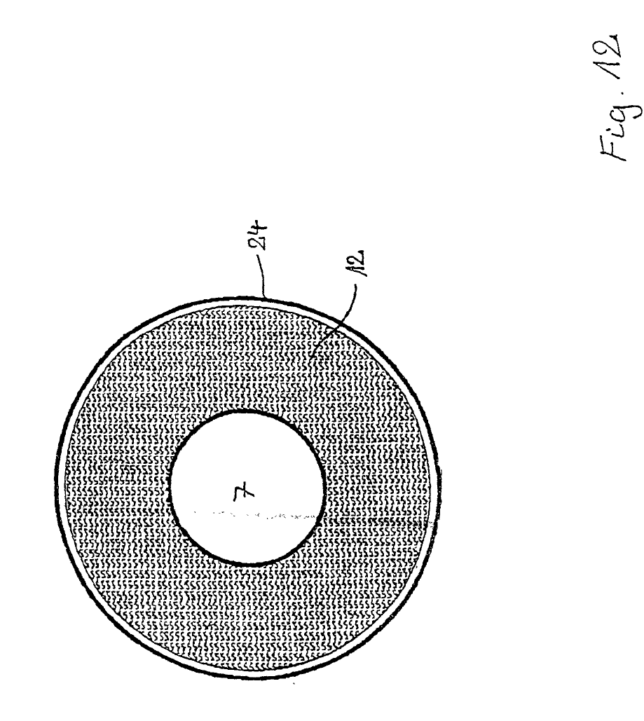

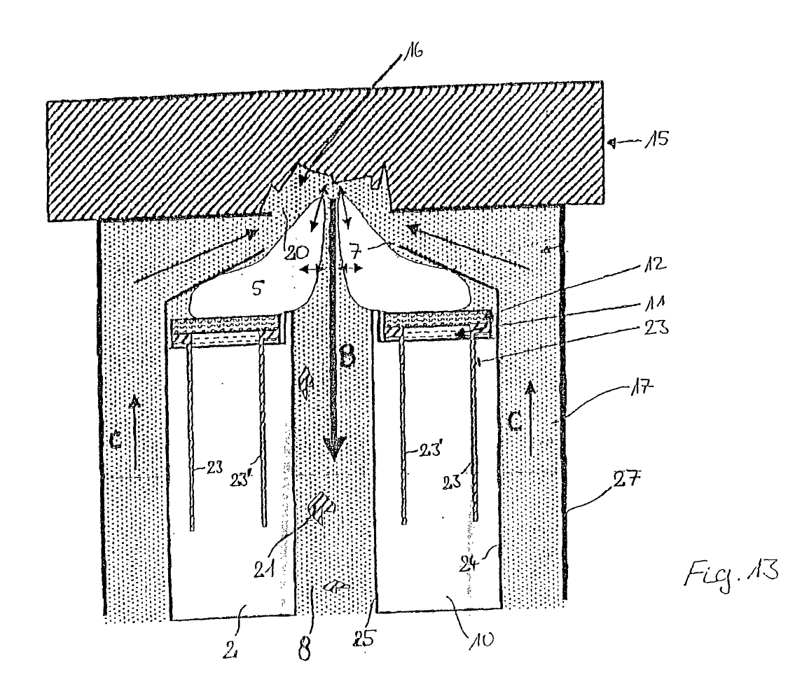

FIG. 12 shows a section along the line A-AA′ in FIG. 11. It can be detected that again the resistance-heated layer 12 is disposed concentrically and thus forms a radially symmetrical vapour bubble. FIG. 13 shows a further example according to the invention with an ablation instrument which copies the one in FIG. 11. In addition to the arrangement in FIG. 11, the tube 2 is now housed in a housing 27 which is disposed concentrically hereto but at a spacing. This housing 27 extends along the external wall 24 of the tube 2 and narrows in a concentric manner inwardly above the opening 7. Said housing consequently forms a further nozzle opening 20 above the opening 7.

In the spacing between the external wall 24 and the housing 27, a capillary system 17 is disposed which serves for supplying evaporation medium to the resistance-heated element 12. The medium flows in the direction of the arrow C into the region between the opening 7 and the nozzle opening 20. Upon heating the resistance-heated element 12, a concentric gas bubble 5 is in turn formed which acts upon the object 15 and fragments the latter there above the nozzle opening which is placed directly on the object 15. A cavity 16 is thereby formed in the medium level 15 and the produced fragments 21 of the removed material are sucked via the suction opening 8 in the direction of the arrow B.

As a result of the fact that the medium to be evaporated in this example is supplied via the channel 17 to the surface of the object 15, it is hence also possible to fragment, to cut or to remove solid bodies.

FIG. 14 shows a plan view of the resistance-heated layer 12 and the electrical contacts 13′, 13″ of a further ablation tool. In the case of this ablation tool, the resistance-heated layer 12 is made of p⁺-doped diamond which is contacted by a p⁺-doped diamond lead and a diamond discharge 13′. These leads and discharges 13′ are in turn connected to metallic contacts 13″ (Cr/Au). The electrically conducting contacts 13′ and the heating element 12 are covered with a nominally undoped diamond layer 14 as passivation. By means of the form of heating element chosen, the ratio of resistances between the heating element 12 and the contacts 13′, 13″ is chosen such that the electrical power is substantially converted in the electrical heating element 12 and there the heat is produced. In particular, the chemically inert and high heat conductive passivation layer 14, which however does not prevent the formation of a vapour bubble above the heating element 12 because of its high heat conductivity, is advantageous with respect to application on biological materials.

We claim:

1. Ablation instrument for cutting, fragmenting and/or removing material of an object, containing a carrier substrate,
having at least one resistance-heating layer which is disposed on the substrate and made of doped diamond or diamond like carbon (DLC) and also at least one electrical lead and at least one electrical discharge which are both electrically connected at different places by the resistance-heated layer.

2. Ablation instrument according to the preceding claim,
characterised in that the substrate contains silicon and/or silicon carbide, silicon oxides (SiO₂, glass), refractory metals or carbides thereof, sapphire, iridium, niobium, tantalum, titanium, tungsten, tungsten carbide, titanium carbide, titanium nitride, silicon nitride, germanium, magnesium oxide, diamond, graphite and/or germanium or is made therefrom.

3. Ablation instrument according to claim 1,
characterised in that the substrate is at least in regions a membrane.

4. Ablation instrument according to on of the preceding claims,
characterised in that the resistance-heated layer has a surface area of less than 10 mm², preferably less than 5 mm².

5. Ablation instrument according to one of the preceding claims,
characterised in that the resistance-heated layer has a dopant concentration greater than or equal to 5×10¹⁷ cm⁻³ and/or up to 10²² cm⁻³.

6. Ablation instrument according to one of the preceding claims,
characterised in that the resistance-heated layer is doped with boron, phosphorus, nitrogen, lithium, hydrogen and/or sulphur.

7. Ablation instrument according to one of the preceding claims,
characterised in that the resistance-heated layer has a thermal power density between 0 and 2 GW/cm³.

8. Ablation instrument according to one of the preceding claims,
characterised in that the resistance-heated layer has a specific resistance between 0.1 μΩcm and 1 Ωcm.

9. Ablation instrument according to the preceding claim,
characterised in that a thermally insulating layer is disposed between the substrate and the resistance-heated layer.

10. Ablation instrument according to the preceding claim,
characterised in that the thermally insulating layer has a heat conductivity of below 1 W·cm⁻¹ K⁻¹ at 300 K.

11. Ablation instrument according to one of the two preceding claims,
characterised in that the thermally insulating layer is made of an amorphous material.

12. Ablation instrument according to one of the three preceding claims,
characterised in that the thermally insulating layer is made of an electrical non-conductor.

13. Ablation instrument according to one of the four preceding claims,
characterised in that the material of the thermally insulating layer is chosen form silicon oxides, silicon nitrides, silicon oxynitrides and aluminium oxides.

14. Ablation instrument according to one of the preceding claims,
characterised in that at least one intermediate layer is disposed between the substrate or the thermally insulating layer and the resistance-heated layer.

15. Ablation instrument according to the preceding claim,
characterised in that the intermediate layer is made of intrinsic or nominally undoped diamond.

16. Ablation instrument according to one of the two preceding claims,
characterised in that the intermediate layer is laterally structured.

17. Ablation instrument according to one of the preceding claims,
characterised in that an electrically insulating layer is disposed at least partially on the resistance-heated layer and/or on the electrical leads and/or discharges.

18. Ablation instrument according to one of the preceding claims,
characterised in that the surface which can be contacted with the object to be treated is hydrogen-terminated, oxygen-terminates, and/or fluorine-terminated.

19. Ablation instrument according to the preceding claim,
characterised in that the carrier substrate is disposed on one end of a stick.

20. Ablation instrument according to the preceding claim,
characterised in that the applicator stick is configured as an applicator tube which has a first opening in the region of a first end and the substrate is disposed in the cavity of the first opening in an adjacent manner such that the surface of the resistance-heated layer is orientated towards the first opening.

21. Ablation instrument according to the preceding claim,
characterised in that the first opening is disposed in an axial direction of the applicator tube or laterally on the circumference of the applicator tube.

22. Ablation instrument according to one of the three preceding claims,
characterised in that the substrate has an external diameter which is smaller than or equal to the internal diameter of the applicator tube or applicator stick and is disposed perpendicularly relative to the axial direction of the applicator tube or the applicator stick.

23. Ablation instrument according to one of the claims 19 to 22,
characterised in that the applicator stick or the applicator tube has a liquid supply and/or a liquid suction removal to or from the surface of the resistance-heated layer.

24. Ablation instrument according to the preceding claim,
characterised in that the liquid supply and/or the liquid suction removal device penetrate through the substrate, the intermediate layer, the thermally insulating layer and the resistance-heated layer as a through-opening.

25. Ablation instrument according to one of the claims 19 to 24,
characterised in that a further internal tube is inserted into the applicator tube and divides the internal space of the applicator tube into two axially extending compartments.

26. Ablation instrument according to one of the claims 19 to 24,
characterised in that a further internal tube is inserted into the applicator tube and divides the internal space of the applicator tube into two concentrically extending compartments.

27. Ablation instrument according to one of the claims 19 to 24,
characterised in that a further internal tube is inserted into the applicator tube and divides the internal space of the applicator tube into two compartments which extend parallel to each other.

28. Ablation instrument according to the preceding claim,
characterised in that both compartments are part of the liquid supply and/or liquid suction removal.

29. Ablation instrument according to one of the preceding claims,
characterised in that the substrate is disposed on the end of the internal tube which is orientated towards the first end of the applicator tube.

30. Ablation instrument according to one of the preceding claims,
characterised in that a nozzle plate is disposed above the resistance-heated layer and has a nozzle opening above the resistance heated layer.

31. Ablation instrument according to one of the preceding claims,
characterised in that a plate is disposed above the resistance-heated layer and has channels for supplying and/or removing by suction liquid to or from the surface of the heating layer.

32. Ablation instrument according to the preceding claim,
characterised in that the channels are configured as capillaries.

33. Ablation instrument according to one of the preceding claims,
characterised in that the electrical lead and discharge have at least one contact material layer, a diffusion barrier disposed thereon and a metallic cover layer disposed on the diffusion barrier.

34. Ablation instrument according to the preceding claim,
characterised in that the contact material layer contains amorphous silicon, Ti, W, Pt, Au, TiW, WC, TiC, TiN, Si, Cu, Be, Fe, Al, Ni, Cr, Sn, Ba, refractory metals and/or doped diamond or is made therefrom.

35. Ablation instrument according to one of the two preceding claims,
characterised in that the diffusion barrier is made of N/W, Ti/Au, C/Au, WC/Au, Ti/Pt/Au and/or conductive diamond-like carbon (DLC).

36. Ablation instrument according to one of the preceding claims,
characterised in that a temperature sensor is disposed in the region of the resistance-heated layer.

37. Ablation instrument according to one of the preceding claims,
characterised in that the resistance layer itself is configured as a temperature sensor.

38. Ablation instrument according to the preceding claim,
characterised in that the temperature sensor is integrated monolithically into the resistance layer.

39. Ablation method for cutting, fragmenting of material or removing material from a surface of an object, wherein a resistance-heated layer made of diamond being brought into direct contact with or into the vicinity of the material to be treated and if necessary a liquid is introduced between the diamond layer and the material to be treated;
the diamond layer is contacted with two electrical conductors and is supplied via the electrical conductors with an electrical current in order to heat it in such a manner that liquid, which is located between the diamond layer and the object and/or the material to be treated, forms vapour bubbles at the contact point to the resistance layer.

40. Ablation method according to the preceding claim,
characterised in that the diamond layer is electrically heated in such a manner that the spinodalian temperature of the liquid is exceeded.

41. Ablation method according to one of the two preceding claims,
characterised in that the resistance-heated layer is brought into contact with or into the vicinity of the surface of the material to be treated or is introduced into the material to be treated.

42. Ablation method according to one of the three preceding claims,
characterized in that the liquid and/or the material removed from the object to be treated is removed by suction.

43. Ablation method according to one of the four preceding claims,
characterised in that an ablation instrument according to one of the claims 1 to 32 is used.

44. Use of an ablation instrument or of an ablation method according to one of the preceding claims for material treatment and shaping, in order to cut, to fragment and/or to remove material, in particular in medicine, in particular in surgery, in eye surgery, in particular for cataract extraction or vitrectomy.
